SEQUENCE LISTING (13 sequences)

SEQ ID NO: 1 - DNA, 35 bp, Artificial Sequence
  PCR Primer
gcggatccatggcaatggtatcagaattcctcaag

SEQ ID NO: 2 - DNA, 33 bp, Artificial Sequence
  PCR Primer
gctctagattagtttcctccacaaagagccacc

SEQ ID NO: 3 - protein, 6 aa, Artificial Sequence
  Extra sequence on N-terminus of recombinant human annexin 1
ISEFGS

SEQ ID NO: 4 - protein, 53 aa, Homo sapiens
MAMVSEFLKQAWFIENEEQEYVQTVKSSKGGPGSAVSPYPTFNPSSDVAALHK

SEQ ID NO: 5 - protein, 44 aa, Homo sapiens
MAMVSEFLKQAWFIENEEQEYVQTVKSSKGGPGSAVSPYPTFNP

SEQ ID NO: 6 - protein, 44 aa, Mus musculus
MAMVSEFLKQARFLENQEQEYVQAVKSYKGGPGSAVSPYPSFNV

SEQ ID NO: 7 - protein, 44 aa, Rattus norvegicus
MAMVSEFLKQACYIEKQEQEYVQAVKSYKGGPGSAVSPYPSFNP

SEQ ID NO: 8 - protein, 44 aa, Sus scrofa
MAMVSEFLKQAWFIDNEEQEYIKTVKGSKGGPGSAVSPYPTFNP

SEQ ID NO: 9 - protein, 44 aa, Bos taurus
MAMVSEFLKQAWFIENEEQEYIKTVKGSKGGPGSAVSPYPTFNP

SEQ ID NO: 10 - protein, 44 aa, Equus caballus
MSMVSAFLKQAWFIENEEQEYIKAVKGSKGGPGSAVSPYPSFNP

SEQ ID NO: 11 - protein, 44 aa, Oryctolagus cuniculus
MAMVSEFLKQAWFIQNEEQDYINTVKTYKGGPGSAVSPYPAFNP

SEQ ID NO: 12 - protein, 40 aa, Columba livia
MAMVSEFLKQAWFMEHQEQEYIKSVKGGPVVPQQQPNFDP

SEQ ID NO: 13 - protein, 39 aa, Gallus gallus
MAMVSEFTKQAWFMDNQEQECIKSSKGGSSVQSRPNFDP

(19) United States
(12) Patent Application Publication
(10) Pub. No.: US 2009/0163578 A1
(43) Pub. Date: Jun. 25, 2009

(54) NOVEL MODIFICATION OF IMMUNOMODULATORY PROTEIN

(75) Inventors: Maxim V. Dorovkov, Piscataway, NJ (US); Alexey G. Ryazanov, Princeton, NJ (US); Lillia V. Ryazanova, Princeton, NJ (US)

Correspondence Address: FOX ROTHSCHILD LLP;PRINCETON PIKE CORPORATE CENTER, 2000 Market Street, Tenth Floor, Philadelphia, PA, 19103, US

(73) Assignee: UNIVERSITY OF MEDICINE AND DENTISTRY OF NEW JERSEY, New Brunswick, NJ (US)

(21) Appl. No.: 11/576,294

(22) PCT Filed: Oct. 3, 2005
(86) PCT No.: PCT/US05/35418

§ 371 Date: Jan. 9, 2009

Related U.S. Application Data
(60) Provisional application No. 60/615,293, filed on Oct. 1, 2004.

(51) Int. Cl. A61K 31/35; C12N 5/06; A01N 1/02

(52) U.S. Cl. 514/456; 435/375; 435/1.1

(57) ABSTRACT

Methods of inhibiting annexin I induced apoptosis by contacting a cell population containing a TRPM7/ChaK1 kinase with an effective amount of a composition containing an inhibitor for the kinase.

### CROSS-REFERENCE TO RELATED APPLICATIONS

This application claims priority under 35 U.S.C. § 119(e) to U.S. Provisional Application Ser. No. 60/615,293, which was filed on Oct. 1, 2004. The disclosure of this application is incorporated herein by reference.

### BACKGROUND OF THE INVENTION

Organisms eliminate unwanted cells by a process variously known as regulated cell death, programmed cell death or apoptosis. Such cell death occurs as a normal aspect of animal development as well as in tissue homeostasis and aging (Glucksmann, A., Biol. Rev. Cambridge Philos. Soc. 26:59-86 (1951); Glucksmann, A., Archives de Biologie 76:419-437 (1965); Ellis et al., Dev. 112:591-603 (1991); Vaux et al., Cell 76:777-779 (1994)). Apoptosis regulates cell number, facilitates morphogenesis, removes harmful or otherwise abnormal cells and eliminates cells that have already performed their function. Additionally, apoptosis occurs in response to various physiological stresses, such as hypoxia or ischemia (PCT published application WO96/20721).

There are a number of morphological changes shared by cells experiencing regulated cell death, including plasma and nuclear membrane blebbing, cell shrinkage (condensation of nucleoplasm and cytoplasm), organelle relocalization and compaction, chromatin condensation and production of apoptotic bodies (membrane enclosed particles containing intracellular material) (Orrenius, S., J. Internal Medicine 237:529-536 (1995)).

Apoptosis is achieved through an endogenous mechanism of cellular suicide (Wyllie, A. H., in Cell Death in Biology and Pathology, Bowen Lockshin, eds., Chapman and Hall (1981), pp. 9-34). A cell activates its internally encoded suicide program as a result of either internal or external signals. The suicide program is executed through the activation of a carefully regulated genetic program (Wylie et al., Int. Rev. Cyt. 68: 251 (1980); Ellis et al., Ann. Rev. Cell Bio. 7: 663 (1991)).

However, excessive cell death may result in crippling degenerative disorders such as Alzheimer's disease, Huntington's Disease, and Parkinson's Disease. Therefore, a need exists to for reliable methods to treat disorders associated with excessive cell death.

### SUMMARY OF THE INVENTION

This need is met by the present invention.

There is provided, in accordance with the present invention, a method of inhibiting annexin I induced apoptosis by contacting a cell population containing a TRPM7/ChaK1 kinase with an effective amount of a composition containing an inhibitor for the kinase. In one embodiment, the inhibitor is rottlerin.

In another embodiment, the cell population is the cell population is a cell line used in industrial biology. In yet another embodiment, the cell population is a transplantation organ.

Also provided is a method of treating a disorder characterized by abnormal cell death induced by annexin I in a patient by administering to the patient a therapeutically effective amount of a composition containing an inhibitor for TRPM7/ChaK1 kinase. In one embodiment, the disorder is a neurodegenerative disorder, heart disease, a retinal disorder, an autoimmune disorder, polycystic kidney disease, or an immune system disorder. In another embodiment, the neurodegenerative disorder is Alzheimer's disease, Huntington's Disease, a prion disease, Parkinson's Disease, multiple sclerosis, amyotrophic lateral sclerosis, ataxia telangiectasia, or spinobulbar atrophy.

Finally, in another embodiment, there is also provided a method for preventing the rejection of a transplanted organ in a patient receiving that organ by administering to that patient an effective amount of a composition containing an inhibitor for TRPM/ChaK1 kinase. In this embodiment the transplanted organs include a heart, a kidney, a pancreas, lungs, a liver, or intestines.

### BRIEF DESCRIPTION OF THE DRAWINGS

FIGS. 1A-E illustrate annexin 1 phosphorylation by ChaK1. Phosphorylated proteins were analyzed by SDS-PAGE or two-dimensional electrophoresis on TLC plates and subsequent autoradiography;

FIG. 1A shows the phosphorylation by ChaK1 of the following proteins: lane 1, proteins in fraction 2 after fractionation of C₂C₁₂ cell lysates; lane 3, human recombinant (recomb.) annexin 1; lane 4, bovine annexin 1. Lane 2, autophosphorylation of ChaK1;

FIG. 1B depicts the time course of annexin 1 phosphorylation by ChaK1;

FIG. 1C is a phosphoamino acid analysis of annexin 1 phosphorylated by ChaK1. Phosphoamino acid analysis was performed by hydrolysis of phosphoproteins with HCl, separation of amino acids using two-dimensional electrophoresis on TLC plates, and autoradiography;

FIG. 1D shows the effect of Ca²⁺ and EGTA on annexin 1 phosphorylation by ChaK1;

FIG. 1E illustrates the phosphorylation of annexin 1 in crude lysates from cells overexpressing TRPM7/ChaK1. HEK293 cells with tetracycline (Tet)-regulatable expression of TRPM7/ChaK1 were incubated with (lanes 2 and 4) or without (lanes 1 and 3) tetracycline. The cell lysates were incubated with [γ-³³P]ATP in phosphorylation mixture with (lanes 3 and 4) or without (lanes 1 and 2) addition of recombinant (recomb.) human annexin 1. The arrow indicates the position of the 210-kDa band that most likely represents the autophosphorylated TRPM7/ChaK1;

FIGS. 2A-D. illustrate the identification of site of phosphorylation in annexin 1;

FIG. 2A shows human recombinant annexin 1 was phosphorylated by ChaK1 and digested with different concentrations of trypsin as described under Examples. Samples were analyzed by SDS-PAGE and autoradiography (Autorad.);

FIG. 2B depicts the amino acid sequence of the N-terminal region of human annexin 1;

FIG. 2C illustrates the results when alanines were substituted for serines in human recombinant annexin 1. Four mutants were produced: (i) S5A, (ii) S27A,S28A, (iii) S34A,S37A, (iv) S45A,S46A. The wild type (WT) and resulting mutant recombinant proteins were phosphorylated by ChaK1. Samples were analyzed by SDS-PAGE and autoradiography;

FIG. 2D illustrates the digestion of phosphorylated annexin 1 with cathepsin D. Human recombinant (recomb.) and bovine annexin 1 were phosphorylated by ChaK1 and digested with cathepsin D in the presence or absence of pepstatin A. Samples were analyzed by SDS-PAGE and autoradiography (Autorad.);

FIG. 3A shows the alignment of the N-terminal regions of annexin 1 from different species. The sequences were obtained from NCBI data bank and aligned using CLUSTAL W (1.60) and BoxShade programs;

FIG. 3B depicts the location of Ser5 (indicated by arrow) in the complex between the N-terminal α-helix of annexin 1 and S100A11 (28), Protein Data Bank number 1QLS;

FIG. 4 depicts circular dichroism spectra of the N-terminal peptides of annexin 1;

FIGS. 5A-K show the effect of TRPM7 overexpression and substitution of Ser5 with Ala or Asp in annexin 1 on cell viability. After transduction with lentiviral based vector containing wt or mutant forms of annexin 1, cells were incubated in the presence (A-E) or absence (F-J) of tetracycline. (A, F) original HEK293-TRPM7tet cell line (B, G) cells transducted with the lentiviral vector containing GFP; (C, H) cells transducted with wild type annexin 1; (D, I) cells transducted with annexin 1 in which Ala was substituted for Ser5, (E, J) cells transducted with annexin 1 in which Asp was substituted for Ser5. (A-J) Cells were visualized by light microscopy, photographed and (K) subsequently analyzed using MTT assay;

FIGS. 6A-C illustrate the effect of monovalent metal ions and protein kinase inhibitors on ChaK1-cat activity;

FIG. 6A is a graph depicting the results when purified recombinant ChaK1-cat was incubated with myelin basic protein in a reaction mixture containing 4 mM MnCl₂, [γ-³³P]ATP, and different concentrations of K⁺ or Na⁺. Kinase reactions were carried out as described in the Examples. The samples were analyzed by SDS-PAGE and autoradiography. The graph was obtained by the quantification of the bands corresponding to phosphorylated myelin basic protein on the autoradiogram using the Kodak 1D imaging program;

FIG. 6B shows the effect of different concentrations of rottlerin or staurosporine on ChaK1-cat activity. The reaction was performed using purified recombinant ChaK1-cat and myelin basic protein. The samples were analyzed by SDS-PAGE and autoradiography; and

FIG. 6C is a graph showing the effect of various concentrations of rottlerin on ChaK1-cat activity. The bands corresponding to phosphorylated myelin basic protein (on the autoradiogram shown in B) were quantified using the Kodak 1D imaging program.

### DETAILED DESCRIPTION OF THE INVENTION

The present invention derives from the discovery that annexin 1 is a substrate for TRPM7/ChaK1. To reflect the bifunctional nature of TRPM7 molecule it is referred to as TRPM7/ChaK1 (Channel-Kinase 1). Further, the terms ChaK1 and ChaK1-cat are used interchangeably throughout the application.

TRPM7/ChaK1 is a member of TRPM family of the TRP superfamily of cation channels. Annexin 1 is a Ca²⁺- and phospholipid-binding protein that can promote Ca²⁺-dependent membrane fusion. Annexin 1 was originally discovered as a mediator of the anti-inflammatory actions of glucocorticoids and was also implicated in the regulation of cell growth and differentiation and apoptosis.

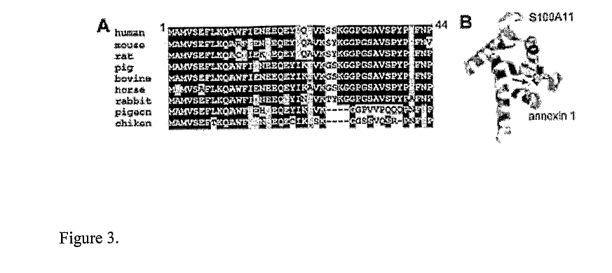

TRPM7/ChaK1 phosphorylates annexin 1 at a conserved serine residue (Ser5) located within the N-terminal amphipathic α-helix. The N-terminal region plays a crucial role in interaction of annexin 1 with other proteins and membranes. The phosphorylation of Ser5 by any protein kinase has not been previously reported and it is therefore specific for TRPM7/ChaK1 protein kinase. In contrast to the sites in annexin 1 that are phosphorylated by other protein kinases, Ser5 is absolutely conserved in mammalian and avian annexin 1 (see FIG. 3A). The phosphorylation of annexin 1 by TRPM7 kinase can modulate the function of annexin 1 in apoptosis.

That is, annexin 1 induced apoptosis can be inhibited by contacting a cell population containing a TRPM7/ChaK1 kinase with an effective amount of a composition containing an inhibitor for the kinase. A preferred inhibitor includes rottlerin.

Methods for inhibiting annexin 1 induced apoptosis can be performed in vivo or in vitro. For example, either a cell line used in industrial biology or a transplantation organ can comprise the cell population.

Accordingly, a disorder characterized by abnormal cell death induced by annexin 1 in a patient can be treated by administering to the patient a therapeutically effective amount of a composition containing an inhibitor for TRPM7/ChaK1 kinase. Suitable disorders include, but are not limited to, neurodegenerative disorders, heart diseases, retinal disorders, autoimmune disorders, polycystic kidney disease, and immune system disorders. Specific neurodegenerative disorders include Alzheimer's disease, Huntington's Disease, prion diseases, Parkinson's Disease, multiple sclerosis, amyotrophic lateral sclerosis, ataxia telangiectasia, and spinobulbar atrophy. Specific heart diseases include myocardial infarction, congestive heart failure and cardiomyopathy. Autoimmune disorders include lupus erythematosus, rheumatoid arthritis, type I diabetes, Sjögren's syndrome and glomerulonephritis. The methods are also useful for reducing or preventing cell, tissue, and organ damage during transplantation; reducing or preventing cell line death in industrial biotechnology; reducing or preventing alopecia (hair loss); and reducing the premature death of skin cells.

In practice, a composition containing an inhibitor for TRPM7/ChaK1 kinase may be administered in any variety of suitable forms, for example, by inhalation, topically, parenterally, rectally or orally; more preferably orally. More specific routes of administration include intravenous, intramuscular, subcutaneous, intraocular, intrasynovial, colonical, peritoneal, transepithelial including transdermal, ophthalmic, sublingual, buccal, dermal, ocular, nasal inhalation via insufflation, and aerosol.

A composition containing an inhibitor for TRPM7/ChaK1 kinase may be presented in forms permitting administration by the most suitable route. The invention also relates to administering pharmaceutical compositions containing at least one inhibitor for TRPM7/ChaK1 which are suitable for use as a medicament in a patient. These compositions may be prepared according to the customary methods, using one or more pharmaceutically acceptable adjuvants or excipients. The adjuvants comprise, inter alia, diluents, sterile aqueous media and the various non-toxic organic solvents. The compositions may be presented in the form of oral dosage forms, or injectable solutions, or suspensions.

The choice of vehicle and the content of TRPM7/ChaK1 inhibitor in the vehicle are generally determined in accordance with the solubility and chemical properties of the product, the particular mode of administration and the provisions to be observed in pharmaceutical practice. When aqueous suspensions are used they may contain emulsifying agents or agents which facilitate suspension. Diluents such as sucrose, ethanol, polyols such as polyethylene glycol, propylene glycol and glycerol, and chloroform or mixtures thereof may also be used. In addition, the TRPM7/ChaK1 inhibitor may be incorporated into sustained-release preparations and formulations.

For parenteral administration, emulsions, suspensions or solutions of the compounds according to the invention in vegetable oil, for example sesame oil, groundnut oil or olive oil, or aqueous-organic solutions such as water and propylene glycol, injectable organic esters such as ethyl oleate, as well as sterile aqueous solutions of the pharmaceutically acceptable salts, are used. The injectable forms must be fluid to the extent that it can be easily syringed, and proper fluidity can be maintained, for example, by the use of a coating such as lecithin, by the maintenance of the required particle size in the case of dispersion and by the use of surfactants. Prolonged absorption of the injectable compositions can be brought about by use of agents delaying absorption, for example, aluminum monostearate and gelatin. The solutions of the salts of the products according to the invention are especially useful for administration by intramuscular or subcutaneous injection. Solutions of the TRPM7/ChaK1 inhibitor as a free base or pharmacologically acceptable salt can be prepared in water suitably mixed with a surfactant such as hydroxypropyl-cellulose. Dispersion can also be prepared in glycerol, liquid polyethylene glycols, and mixtures thereof and in oils. The aqueous solutions, also comprising solutions of the salts in pure distilled water, may be used for intravenous administration with the proviso that their pH is suitably adjusted, that they are judiciously buffered and rendered isotonic with a sufficient quantity of glucose or sodium chloride and that they are sterilized by heating, irradiation, microfiltration, and/or by various antibacterial and antifungal agents, for example, parabens, chlorobutanol, phenol, sorbic acid, thimerosal, and the like.

Sterile injectable solutions are prepared by incorporating the TRPM7/ChaK1 inhibitor in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

Topical administration, gels (water or alcohol based), creams or ointments containing the TRPM7/ChaK1 inhibitor may be used. The TRPM7/ChaK1 inhibitor may be also incorporated in a gel or matrix base for application in a patch, which would allow a controlled release of compound through transdermal barrier.

For administration by inhalation, the TRPM7/ChaK1 inhibitor may be dissolved or suspended in a suitable carrier for use in a nebulizer or a suspension or solution aerosol, or may be absorbed or adsorbed onto a suitable solid carrier for use in a dry powder inhaler.

The percentage of TRPM7/ChaK1 kinase inhibitor in the compositions used in the present invention may be varied, it being necessary that it should constitute a proportion such that a suitable dosage shall be obtained. Obviously, several unit dosage forms may be administered at about the same time. A dose employed may be determined by a physician or qualified medical professional, and depends upon the desired therapeutic effect, the route of administration and the duration of the treatment, and the condition of the patient. In the adult, the doses are generally from about 0.001 to about 50, preferably about 0.001 to about 5, mg/kg body weight per day by inhalation, from about 0.01 to about 100, preferably 0.1 to 70, more especially 0.5 to 10, mg/kg body weight per day by oral administration, and from about 0.001 to about 10, preferably 0.01 to 10, mg/kg body weight per day by intravenous administration. In each particular case, the doses are determined in accordance with the factors distinctive to the patient to be treated, such as age, weight, general state of health and other characteristics which can influence the efficacy of the compound according to the invention.

The TRPM7/ChaK1 kinase inhibitor used in the invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the TRPM7/ChaK1 kinase inhibitor may be administered 1 to 4 times per day. Of course, for other patients, it will be necessary to prescribe not more than one or two doses per day.

The following non-limiting examples set forth hereinbelow illustrate certain aspects of the invention.

### EXAMPLES

### Overview

TRPM7/ChaK1 kinase domain (ChaK1) was expressed in bacteria and analyzed in detail the activity of purified kinase. To identify substrates for TRPM7/ChaK1, cell lysate fractionation, phosphorylation with purified recombinant ChaK1, and subsequent peptide mass fingerprinting by MALDI-TOF mass spectrometry were used. By analysis with antibodies against TRPM7/ChaK1 of various cell lines we found the highest level of TRPM7/ChaK1 in C₂C₁₂ mouse myoblasts (data not shown).

### Materials:

Chemicals were obtained from Sigma. Rottlerin and staurosporine were dissolved in Me₂SO. Radioisotopes were from PerkinElmer Life Sciences. Expression and purification of recombinant ChaK1 kinase domain was performed as described (. Ryazanova, L. V., Dorovkov, M. V., Ansari, A., and Ryazanov, A. G. (2004) J. Biol. Chem. 279, 3708-16.). Bovine annexin 1 was from Biodesign. Human annexin V was from Sigma. Human recombinant annexin II was a kind gift of Dr. Valery Alakhov (Supratek Pharma, Inc.).

### Example 1

### Fractionation of Cell Lysates and Analysis of Fractions for Phosphorylated Proteins

Mouse C₂C₁₂ cells were collected by trypsinization, washed with ice-cold phosphate-buffered saline, and lysed using Dounce homogenizer in ice-cold buffer containing 30 mM Tris-HCl (pH 8.0), 20 mM NaCl, 1 mM MgCl₂, 1 mM EDTA, 800 μl/L β-mercaptoethanol, 5% glycerol (w/v), complete protease inhibitor (Roche), and 1 mM phenylmethylsulfonyl fluoride. The lysates were cleared twice by centrifugation at 30,000×g for 30 min at 4° C. The cleared lysate (containing 20 mg of total protein) was then fractionated by fast protein liquid chromatography on Mono Q HR 5/5 column (Amersham Biosciences) using 20-500 mM NaCl gradient. 40 fractions were collected (1 ml each). 10 μl of each fraction were incubated with [γ-³³P]ATP in phosphorylation mixture (as described below) with or without the addition of recombinant ChaK1. C₂C₁₂ cell lysate was fractionated by chromatography on Mono Q column using 20-500 mM NaCl gradient.

### Example 2

### Protein Phosphorylation Assay

Protein samples were incubated in phosphorylation mixture consisting of 50 mM HEPES-KOH (pH 7.4), 10 mM MgCl₂, 4 mM MnCl₂, 0.5 mM CaCl₂ (unless stated otherwise), 100 μM ATP, and 2 μCi of [γ-³³P]ATP (specific activity of 3000 Ci/mmol) with 0.1 μg of purified recombinant ChaK1. In assays involving annexin 0.5 μg of annexin was used. The reactions were run at 30° C. for 5 min and were terminated by incubation in an ice/water bath and addition of Laemmli sample buffer. Samples were boiled for 5 min and analyzed by SDS-PAGE and autoradiography.

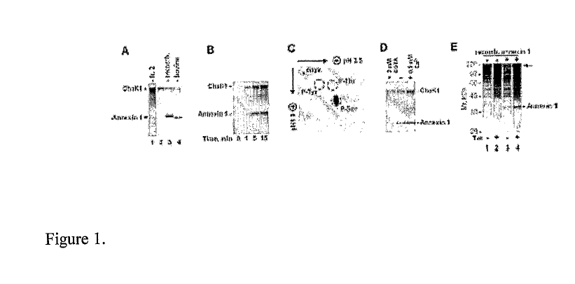

A sample from each fraction was incubated with [γ-³³P]ATP in phosphorylation mixture with or without addition of purified recombinant ChaK1. Fraction number 2 contained a polypeptide with the molecular mass of ˜37 kDa that was intensively phosphorylated by ChaK1 (FIG. 1A).

### Example 3

### Preparation of Samples for MALDI-TOF Analysis

Coomassie-stained polypeptide was excised from the SDS-PAGE gel and digested with trypsin as described (Jimenez, C. R., Huang, L., Qiu, Y., and Burlingame (1998) Current Protocols in Protein Science 16.3.1-16.3.6., John Wiley & Sons, Inc., USA). The samples were prepared according to manufacturers protocol (Applied Biosystems). The samples were analyzed using mass spectrometer Voyager-DE PRO Workstation (Applied Biosystems) in reflector mode. The obtained monoisotopic peptide masses were run against NCBI and Swiss-Prot databases using MS-Fit (Protein Prospector) and PetIdent programs.

The Coomassie-stained 37-kDa polypeptide was excised from the gel in Example 1 and subjected to digestion with trypsin as described in Example 6. The resulting peptides were analyzed by MALDI-TOF mass spectrometry. The obtained masses of tryptic peptides were scanned against NCBI and Swiss-Prot protein data bases using MS-Fit (Protein Prospector) and PeptIdent programs. The set of peptide masses matched to annexin 1.

### Example 4

### Expression and Purification of Annexin 1

Human annexin 1 was expressed as a fusion with maltose-binding protein in Escherichia coli. A DNA fragment corresponding to annexin 1 was produced by PCR using HeLa marathon ready cDNA library (Clontech) and the following primers: GCGGATCCATGGCAATGGTATCAGAATTCCTCAAG (containing BamHI restriction site) and GCTCTAGATTAGTTTCCTCCACAAAGAGCCACC (containing XbaI restriction site). The PCR fragment was inserted into a pMAL-c2x vector (New England Biolabs) using BamHI and XbaI restriction sites to produce the pMcAn1-4 expression construct. Expression and purification of annexin 1 was performed as described for the ChaK1-cat (long form) (Ryazanova, L. V., Dorovkov, M. V., Ansari, A., and Ryazanov, A. G. (2004) J. Biol. Chem. 279, 3708-3716). The resulting fusion protein was cleaved with 2 μg/ml of Factor Xa (New England Biolabs) for 24 h at room temperature to remove maltose binding protein tag from annexin 1. After cleavage, annexin 1 contained 6 additional amino acids on its N terminus (Ile-Ser-Glu-Phe-Gly-Ser).

To confirm that phosphorylated protein of 37 kDa was annexin 1, we analyzed whether recombinant human annexin 1 and purified bovine annexin 1 can be phosphorylated by ChaK1. Indeed, we found that both recombinant human annexin 1 and bovine annexin 1 were phosphorylated by ChaK1 (FIG. 1A).

### Example 5

### Phosphoamino Acid Analysis

Phosphorylation of annexin 1 was performed as described above. Sample preparation was performed as described (Ryazanova, L. V., Dorovkov, M. V., Ansari, A., and Ryazanov, A. G. (2004) J. Biol. Chem. 279, 3708-3716). Phosphoamino acids were separated by two-dimensional electrophoresis on TLC plates 10×10 cm (cellulose on glass, Merck). First dimension was performed in pH 1.9 electrophoresis buffer containing 0.58 M formic acid and 1.36 M acetic acid at 1000 V for 20 min and second dimension in pH 3.5 electrophoresis buffer containing 0.87 M acetic acid, 0.5% (v/v) pyridine, and 0.5 mM EDTA at 1000 V for 8 min. The TLC plates were stained with 0.2% ninhydrin in ethanol and exposed to x-ray film (Eastman Kodak Co.).

Next, we analyzed time dependence of annexin 1 phosphorylation by ChaK1 (FIG. 1B) and performed phosphoamino acid analysis of phosphorylated annexin 1 (FIG. 1C). We found that ChaK1 phosphorylates annexin 1 exclusively on serine residues. Since annexin 1 is a Ca²⁺-regulated protein, we analyzed the effect of Ca²⁺ on phosphorylation of annexin 1 by ChaK1. We found that Ca²⁺ significantly stimulated phosphorylation of annexin 1, while addition of 2 mM EGTA reduced this phosphorylation (FIG. 1D). We also examined whether other members of the annexin family, annexin II and annexin V, can be phosphorylated by ChaK1. No phosphorylation was detected (data not shown) indicating that phosphorylation activity of ChaK1 is specific for annexin 1.

We further investigated phosphorylation of annexin 1 in crude lysates obtained from cells overexpressing TRPM7/ChaK1. We used HEK293 cell line, in which TRPM7/ChaK1 expression can be induced by addition of tetracycline. We found that phosphorylation of annexin 1 was greatly increased in lysates obtained from cells overexpressing TRPM7/ChaK1 (FIG. 1E). This indicates that annexin 1 can be phosphorylated by native full-length TRPM7/ChaK1. We also observed intensively phosphorylated 210-kDa band in cell extracts overexpressing TRPM7/ChaK1 (see FIG. 1E, lanes 2 and 4). This band most likely represents autophosphorylated TRPM7/ChaK1.

### Example 6

### Determination of the Site of Annexin 1 Phosphorylation by ChaK1

Phosphopeptide Mapping—Phosphorylated protein was excised from the SDS-PAGE gel. The protein was digested with trypsin as described (Boyle, W. J., van der Geer, P., and Hunter, T. (1991) Methods Enzymol. 201, 110-149). The obtained peptides were resolved by two-dimensional separation on TLC plates (Merck). In the first dimension peptides were separated by electrophoresis for 7 min at 1 kV in pH 1.9 buffer containing 0.58 M formic acid and 1.36 M acetic acid and in second dimension by ascending chromatography with n-butanol/pyridine/glacial acetic acid/H₂O, 75:50:15:60 (v/v). The phosphopeptides were detected by autoradiography.

Digestion of Annexin 1 with Trypsin and Cathepsin D—Phosphorylated protein was incubated with different amounts of sequence grade modified trypsin (Promega) for 15 min at 37° C. The reactions were stopped by addition of 60 μg/ml of soybean trypsin inhibitor (Sigma). Samples were diluted with Laemmi sample buffer and boiled. The samples were analyzed by SDS-PAGE and autoradiography.

Phosphorylated proteins were digested with 2 μg of cathepsin D (Sigma) in 50 mM Tris acetate (pH 4.5) (50 μl of total reaction volume) for 30 min at 37° C. As a control the same reactions were carried out in the presence of 2 μM of pepstatin A (Sigma). The reactions were stopped by boiling the samples in Laemmli sample buffer. The samples were analyzed by SDS-PAGE and autoradiography.

Site-directed Mutagenesis and Expression of Mutant Proteins—Site directed mutagenesis was performed with QuikChange XL mutagenesis kit (Stratagene) in accordance with manufacturer's protocol using the pMcAn1-4 expression construct as a template. The wild type and mutant annexin 1 were expressed and purified as described above.

Annexin 1 was phosphorylated by ChaK1 and subjected to complete trypsin digestion, with subsequent two-dimensional separation of phosphopeptides on TLC plates. We detected one major phosphopeptide indicating that annexin 1 contains one major site of phosphorylation for ChaK1 (data not shown).

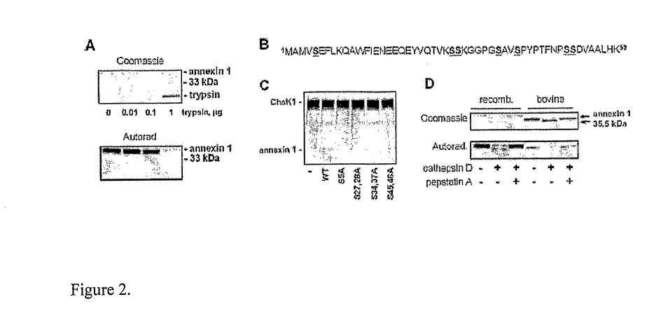

To locate the site of phosphorylation within annexin 1, partial proteolysis of phosphorylated annexin 1 was performed using different concentrations of trypsin. Annexin 1 contains a dense core and a flexible N-terminal region, which could be removed by partial proteolysis. The partial proteolysis produced a band of ˜33 kDa that did not retain radioactive label (FIG. 2A), suggesting that the site of phosphorylation is located within the N-terminal region of annexin 1. The region of annexin 1, which could be cleaved off by trypsin, contains 7 serine residues that could possibly be phosphorylated by ChaK1 (FIG. 2B). Four mutants (one protein with single mutation and three proteins with two mutations) in which Ala was substituted for Ser were produced using QuikChange XL mutagenesis kit (Stratagene). The following mutants were produced: (i) S5A, (ii) S27A,S28A, (iii) S34A,S37A, and (iv) S45A,S46A. The wild type and mutant proteins were expressed in bacteria, affinity-purified, and analyzed for phosphorylation with ChaK1. We found that S5A substitution dramatically reduced phosphorylation of annexin 1 by ChaK1, while intensity of phosphorylation of other mutants were similar to wild type protein (FIG. 2C), suggesting that ChaK1 phosphorylates annexin I at Ser5. To confirm the location of phosphorylated residue in annexin 1, phosphorylated annexin 1 was digested with cathepsin D, which has been shown to cleave annexin 1 specifically at Trp¹² producing a band with molecular mass of ˜35.5 kDa. In this digestion experiment we used human recombinant annexin 1 as well as purified bovine annexin 1. Treatment of phosphorylated human recombinant or bovine annexin 1 with cathepsin D produced a 35.5-kDa band, which lost virtually all radioactive label (FIG. 2D). As a control, to account for possible phosphatase activity in the reaction, the treatment of annexin 1 with cathepsin D was carried out in the presence of pepstatin A (an inhibitor of cathepsin D). In the presence of pepstatin A, annexin 1 was not cleaved and remained radioactively labeled (FIG. 2D). Therefore, we found that ChaK1 phosphorylates annexin 1 specifically at Ser5. This serine residue is evolutionarily conserved and present in all mammalian and avian species (FIG. 3A). Ser5 is located within the N-terminal α-helix, which specifically interacts with S100A11 protein (FIG. 3B).

### Example 7

### Effects of the Phosphorylation of Ser5 on the N-Terminal α-Helix of Annexin 1

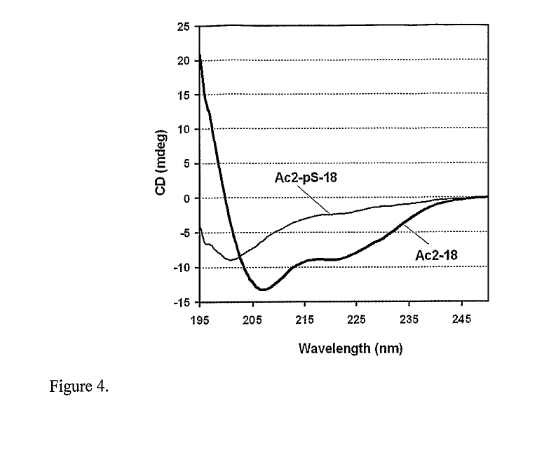

Two N-acetylated peptides were used: the peptide corresponding to the N-terminal region of annexin 1 (Ac2-18) and the peptide with the phospho-Ser5 (Ac2-pSer-18) (FIG. 4). CD spectra of the peptides (0.01 mg/ml) in the presence of 10 mM 4,4-dimethyl-4-silapentane-1-sulfonate were acquired from 250 to 195 nm in 1 nm increments. CD measurements were acquired at 25° C. on an Aviv Model 202 spectropolarimeter using 1 cm path-length cell. The CD signal of the solvent was subtracted from the scans.

The phosphorylation of Ser5 has a dramatic effect on the structure N-terminal a-helix of annexin 1. According to CD spectroscopy analysis, in the presence of membrane mimetic, unphosphorylated peptide corresponding to the N-terminal region of annexin 1 has predominantly α-helical structure; however, it becomes predominantly random-coil after phosphorylation at Ser5 (FIG. 4). Since, the structure of the N-terminal region of annexin 1 is important for its interaction with S100A11, membranes and Formyl Peptide Receptors, the obtained data strongly indicate that all these functions of annexin 1 can be affected by phosphorylation at Ser5.

### Example 8

### Effect of Phosphorylation of Ser5 in Annexin 1 on Cell Death/Survival

We used cell line with tetracycline-regulated expression of TRPM7 (“HEK293-TRPM7tet”), which has been already used to study TRPM7 channel properties and was shown to express functional full length TRPM7 molecule upon addition of tetracycline. After transduction with lentiviral based vector containing wt or mutant forms of annexin 1, cells were incubated in the presence (FIGS. A-E) or absence (FIGS. F-J) of tetracycline. (A-J) Cells were visualized by light microscopy, photographed and (K) subsequently analyzed using MTT assay.

Prior to experiment described above we analyzed various cell lines with antibody against annexin 1 and found that HEK293 cell line express barely detectable levels of endogenous annexin 1 and, therefore, could be used for expression of wt or mutant forms of annexin 1. To express annexin 1 or GFP we used lentiviral expression system on the basis of vector pLenti (Invitrogen). GFP was used as a “vector control” as well as a control for the efficiency of viral transduction. According to GFP expression efficiency of viral transduction was more then 90%. Expression of wt and mutant forms of annexin 1 was confirmed by western blot analysis with antibody against annexin 1, same levels of expression of wt and mutant forms of annexin 1 were observed. To prevent detachment of HEK293 cells upon prolonged expression of TRPM7, the cells were grown on plates pretreated with poly-lysine.

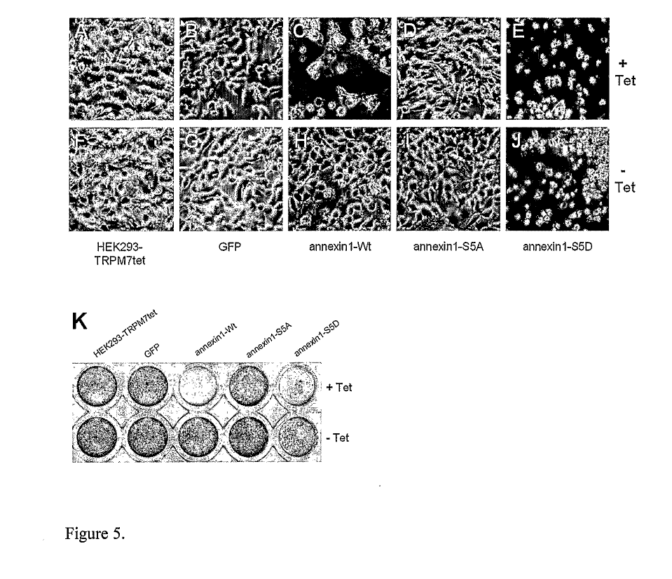

We have recently found that substitution of Ser5 with Ala or Asp in annexin 1 has a dramatic effect on cell viability. We expressed (i) WT human annexin 1, (ii) phosphorylation-deficient mutant of annexin 1 (S5A), or (iii) phosphorylation-mimicking mutant of annexin 1 (S5D) in HEK293 cell line with tetracycline-regulated expression of TRPM7 (“HEK293-TRPM7tet”) (FIG. 5). First, we verified that annexin 1 is phosphorylated by TRPM7 in vivo using metabolic labeling of these cells with ³³Pi, inducing TRPM7 expression with tetracycline and analyzing phosphorylated proteins by 2D-gels (performed in Kendrick Laboratories) and autoradiography. Indeed, we found that phosphorylation of annexin 1 was significantly increased in cells overexpressing TRPM7. Analyzing viability of the cell lines, we found that expression of WT annexin 1 in cells expressing TRPM7 results in cell death (FIG. 5C), however cells survived when phosphorylation-deficient mutant of annexin 1 was expressed (FIG. 5D); expression of phosphorylation-mimicking mutant of annexin 1 resulted in dramatic decrease in cell viability, irrespective of the level of TRPM7 (FIGS. 5E, J). We also used NIH 3T6 cells (which normally express a high level of endogenous TRPM7) and found that expression of annexin 1 in 3T6 cells produced same effects as in “HEK293-TRPM7tet” cells induced with tetracycline: expression of WT as well as phosphorylation-mimicking mutant of annexin 1 in 3T6 cells resulted in cell death, while cells expressing phosphorylation-deficient mutant of annexin 1 survived.

### Example 9

### Analysis of Protein Kinase Inhibitors

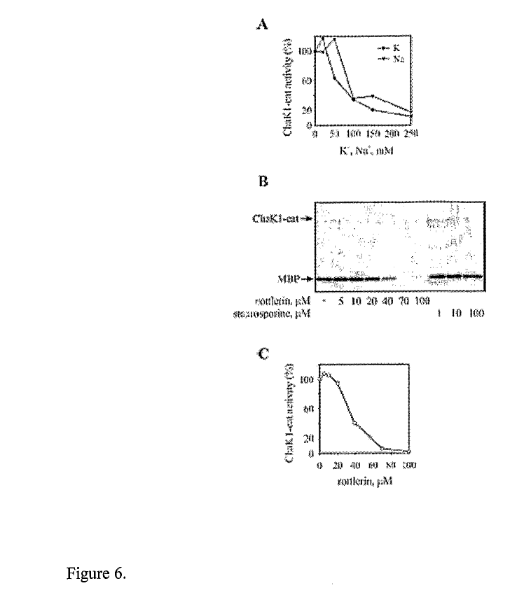

We analyzed the sensitivity of ChaK1 to some known inhibitors of conventional protein kinases. Interestingly, ChaK1 appears to be resistant to staurosporine, which did not produce any inhibitory effect even at the concentration of 100 μM (FIG. 6B). Another protein kinase inhibitor, rottlerin, inhibits ChaK1 with an IC₅₀ of ˜35 μM (FIGS. 6B, C).

We found that staurosporine, a compound that interferes with ATP binding and inhibits most conventional protein kinases, does not have any effect on the kinase activity of ChaK1 at concentrations up to 0.1 mM (FIG. 6B). This result was surprising given the structural similarity between ChaK1 and conventional protein kinases. However, detailed structural analysis suggests an explanation for this result. In conventional protein kinases, there is substantial rearrangement of the residues in the active site to accommodate the bulky staurosporine molecule. However, in ChaK1, there is a salt bridge between Glu-1718 and Lys-1646 in the back of the hydrophobic pocket, which limits the flexibility of the binding site and makes staurosporine binding unlikely. Because amino acids making this salt bridge are conserved in all α-kinases, it is likely that other α-kinases will also not be inhibited by staurosporine. In fact, it was shown previously that eEF-2 kinase is relatively resistant to staurosporine.

Rottlerin, another compound known to inhibit protein kinases, inhibits both autophosphorylation and phosphorylation of myelin basic protein by ChaK1 with an IC₅₀ of ˜35 μM (FIGS. 6B, C).

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims.

What is claimed is:

1. A method of inhibiting annexin I induced apoptosis comprising contacting a cell population containing a TRPM7/ChaK1 kinase with an effective amount of a composition containing an inhibitor for the kinase.

2. The method of claim 1, wherein the inhibitor comprises rottlerin.

3. The method of claim 1, wherein said cell population comprises a cell line used in industrial biology.

4. The method of claim 1, wherein said cell population comprises a transplantation organ.

5. A method of treating a disorder characterized by abnormal cell death induced by annexin I in a patient, said method comprising administering to said patient a therapeutically effective amount of a composition containing an inhibitor for TRPM7/ChaK1 kinase.

6. The method of claim 5, wherein said disorder is a neurodegenerative disorder, heart disease, a retinal disorder, an autoimmune disorder, polycystic kidney disease, or an immune system disorder.

7. The method of claim 6, wherein the neurodegenerative disorder is Alzheimer's disease, Huntington's Disease, a prion disease, Parkinson's Disease, multiple sclerosis, amyotrophic lateral sclerosis, ataxia telangiectasia, or spinobulbar atrophy.

8. A method for preventing the rejection of a transplanted organ in a patient receiving the organ comprising administering to the patient an effective amount of a composition containing an inhibitor for TRPM7/ChaK1 kinase.

9. The method of claim 8, wherein the transplanted organ comprises a heart, a kidney, a pancreas, lungs, a liver, or intestines.
